(19) United States
(12) Patent Application Publication
(10) Pub. No.: US 2015/0223727 A1
(43) Pub. Date: Aug. 13, 2015

(54) CAPSULE WITH STRAIN GAUGE SENSORS TO SENSE EVENTS IN THE GASTROINTESTINAL TRACT

(71) Applicant: CHECK-CAP LTD., Isfiya (IL)

(72) Inventors: Yoav Kimchy, Haifa (IL); Shai Brenner, Haifa (IL)

(73) Assignee: CHECK-CAP LTD., Isfiya (IL)

(21) Appl. No.: 14/425,058

(22) PCT Filed: Jul. 31, 2013
(86) PCT No.: PCT/IL2013/050649

§ 371 Date: Mar. 2, 2015

Related U.S. Application Data
(60) Provisional application No. 61/697,863, filed on Sep. 7, 2012.

(51) Int. Cl. A61B 5/07; A61B 5/06; A61B 1/04; A61B 5/00

(52) U.S. Cl. CPC A61B 5/073; A61B 5/4255; A61B 5/065; A61B 5/0059; A61B 1/041; A61B 5/7282

(57) ABSTRACT

A capsule for examining the gastrointestinal tract, including, a capsule shell enclosing the capsule, wherein said capsule shell is designed to be swallowed by a user to traverse the user's gastrointestinal tract internally, a strain gauge coupled to the capsule shell for measuring strain forces exerted on the capsule shell, a control for receiving the measurements from the strain gauge and responding to the measurements.

### RELATED APPLICATIONS

The present application claims priority from U.S. Provisional application No. 61/697,863 filed on Sep. 7, 2012, the disclosure of which is incorporated herein by reference.

### TECHNICAL FIELD

The present disclosure relates generally to investigating the insides of a patient's colon using an intra-lumen capsule and more specifically to identifying positions of interest based on sensor readings measured by the capsule.

### BACKGROUND

One method for examining the gastrointestinal tract for the existence of polyps and other clinically relevant features that may indicate regarding the potential of cancer is performed by swallowing an imaging capsule that will travel through the gastrointestinal (GI) tract and viewing the patient's situation internally. In a typical case the trip can take between 24-48 hours, after which the imaging capsule exits in the patient's feces. Typically the patient swallows a contrast agent to enhance the imaging ability of the imaging capsule. Then the patient swallows the imaging capsule to examine the gastrointestinal tract while flowing through the contrast agent. The imaging capsule typically includes a radiation source, for example including a radioisotope that emits X-rays or Gamma rays. The radiation is typically collimated to allow it to be controllably directed in a specific direction during the imaging process. In some cases the imaging capsule is designed to measure Compton back-scattering or X-ray florescence and wirelessly transmit the measurements (e.g. a count rate) to an external analysis device, for example a computer or other dedicated instruments.

In a typical implementation a radio-opaque contrast agent is used so that a position with a polyp will have less contrast agent and will measure a larger back-scattering count to enhance accuracy of the measurements. Alternatively, other methods may be used to image the gastrointestinal tract.

U.S. Pat. No. 7,787,926 to Kimchy, the disclosure of which is incorporated herein by reference, describes details related to the manufacture and use of such an imaging capsule.

The use of the imaging capsule exposes the user to radiation, which is potentially harmful. Accordingly, it is of interest to limit the user's exposure to radiation when not necessary, for example by only radiating when needed and blocking the release of radiation from the capsule in locations that do not need to be measured. The imaging capsule may be designed with a concealment mechanism that can be instructed to block radiation when not needed for scanning. Optionally, the concealment mechanism would normally be in the closed position, preventing radiation from exiting the capsule when it is not scanning.

In a typical embodiment it would be desirable that the imaging capsule be instructed to selectively scan with radiation only when movement in the colon occurs, since there is no need to repeatedly scan the same position. The use of selective scanning can also preserve energy, thus prolonging the life of the battery and/or enabling the use of a smaller size battery.

There is thus a need for identifying suspect positions in the colon without activating the radiation scanning mechanism. Additionally it is desirable to sense colon contractions for determining if the capsule is in motion or obstructed to help identify suspect positions and/or be able to decide if to activate the radiation scanning mechanism for producing images of the colon.

### SUMMARY

An aspect of an embodiment of the disclosure relates to a capsule equipped with one or more strain gauges to sense stress and strain forces exerted on the capsule while traveling through the gastrointestinal tract. The capsule is enclosed by a capsule shell to which the strain gauge is coupled. In some embodiments of the disclosure the strain gauge is coupled to the exterior side of the capsule shell. Alternatively the strain gauge is coupled to the interior side with other elements that are inside the capsule. The measurements recorded by the strain gauge are transferred to a control inside the capsule for analysis. The control analyzes the measurements and identifies positions, which are suspected as having obstructions that interfere with the motion of the capsule.

In some embodiments of the disclosure, the capsule may accept measurements from other sensors to increase the accuracy of the analysis. The other sensors may include a pressure sensor, a position tracking system, a 3D accelerometer, a 3D compass, electrodes, an optical sensor or an infra red sensor. Optionally, the other sensors may be active simultaneously with the strain gauge. Alternatively, the other sensors may be activated in response to measurements taken by the strain gauge. In some embodiments of the disclosure, the control may activate a radiation image scanner to scan and provide images of suspect areas responsive to the measurements taken by the strain gauge.

In an exemplary embodiment of the disclosure, the control inside the capsule may communicate wirelessly with an external control, for example executed by a general purpose computer that receives the transmission of information from the internal control. Optionally, the external control can provide instructions to the capsule based on information provided by the internal control.

There is thus provided according to an exemplary embodiment of the disclosure, a capsule for examining the gastrointestinal tract, comprising:
a capsule shell enclosing the capsule; wherein said capsule shell is designed to be swallowed by a user to traverse the user's gastrointestinal tract internally;
a strain gauge coupled to the capsule shell for measuring strain forces exerted on the capsule shell;
a control for receiving the measurements from the strain gauge and responding to the measurements.

In an exemplary embodiment of the disclosure, the capsule further comprises an image scanner that uses radiation to scan images of the surroundings of the capsule;
wherein the image scanner is activated selectively by the control responsive to the measurements from the strain gauge. Optionally, the capsule further comprises multiple strain gauges each monitoring a different area of the capsule shell. In an exemplary embodiment of the disclosure, the control follows movement of strain forces across multiple strain gauges. Optionally, the control identifies obstructions in the gastrointestinal tract responsive to the measurements from the strain gauge. In an exemplary embodiment of the disclosure, the control identifies obstructions in the gastrointestinal tract responsive to the measurements from the strain gauge and at least one other sensor.

In an exemplary embodiment of the disclosure, the at least one other sensor is a pressure sensor that measures hydrostatic pressure due to the content surrounding the capsule. Optionally, the at least one other sensor is a position tracking system. In an exemplary embodiment of the disclosure, the at least one other sensor is an infra-red sensor. In an exemplary embodiment of the disclosure,

the at least one other sensor is a 3D accelerometer. Optionally, the at least one other sensor is a 3D compass. In an exemplary embodiment of the disclosure, the capsule has a specific gravity greater than 1. Optionally, the strain gauge is coupled to the exterior of the shell capsule. Alternatively, the strain gauge is coupled to the interior of the shell capsule.

There is further provided according to an exemplary embodiment of the disclosure, a method of examining the gastrointestinal tract, comprising:
swallowing a capsule enclosed by a capsule shell;
measuring strain forces exerted on the capsule shell with a strain gauge coupled to the capsule shell;
transferring the measurements to a control; and
identifying obstructions from the measurements.

In an exemplary embodiment of the disclosure, the method further comprises selectively activating an image scanner that uses radiation to scan images of the surroundings of the capsule responsive to the measurements from the strain gauge. Optionally, the method further comprises accepting measurements from at least one other sensor. In an exemplary embodiment of the disclosure, the other sensor is a pressure sensor that measures hydrostatic pressure due to the content surrounding the capsule.

### BRIEF DESCRIPTION OF THE DRAWINGS

The present disclosure will be understood and better appreciated from the following detailed description taken in conjunction with the drawings. Identical structures, elements or parts, which appear in more than one figure, are generally labeled with the same or similar number in all the figures in which they appear, wherein:

FIG. 1 is a schematic illustration of a capsule with a strain gauge mounted on the shell of the capsule, according to an exemplary embodiment of the disclosure;

FIGS. 2B-2J are schematic illustrations of capsules with a strain gauge having various shapes and in various positions, according to an exemplary embodiment of the disclosure;

FIGS. 3A-3C are schematic illustrations of capsules with various strain forces applied to them, according to an exemplary embodiment of the disclosure;

FIG. 4 is a schematic illustration of a capsule with an array of strain gauges mounted on the internal or external surface of the capsule shell, according to an exemplary embodiment of the disclosure;

FIG. 5 is a schematic illustration of a capsule colliding with an obstruction, according to an exemplary embodiment of the disclosure;

FIG. 6 is a schematic illustration of a capsule sliding over an obstruction, according to an exemplary embodiment of the disclosure; and

FIG. 7 is a schematic illustration of a capsule interacting with obstructions in a colon versus the readings of a strain gauge, according to an exemplary embodiment of the disclosure.

### DETAILED DESCRIPTION

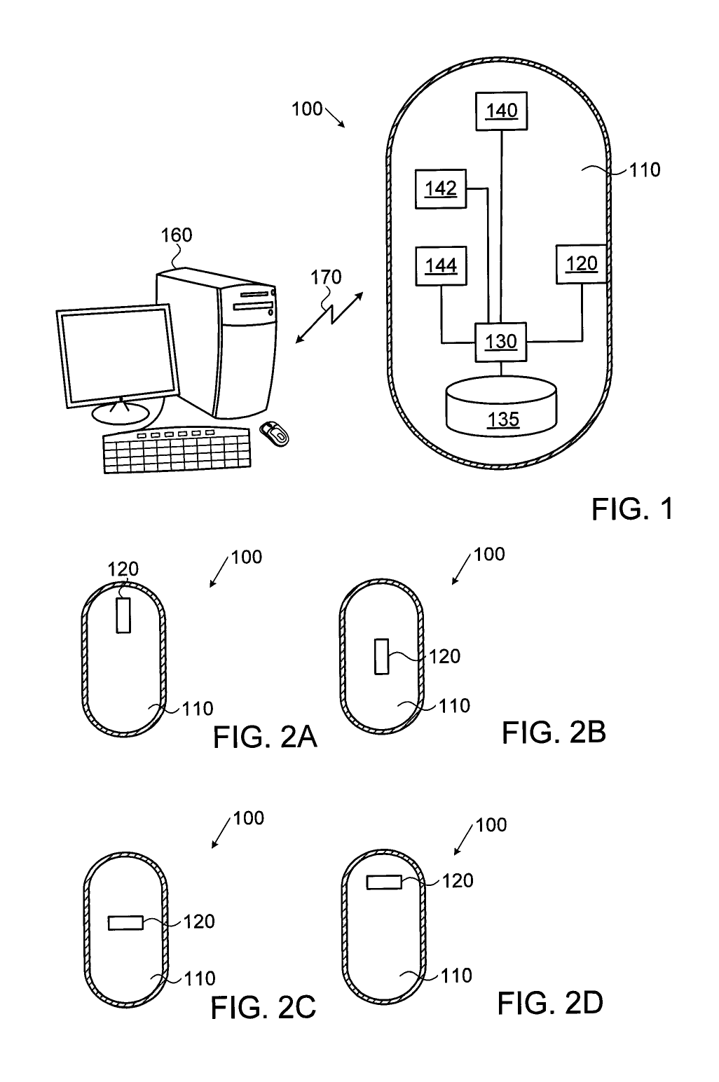

In an exemplary embodiment of the disclosure, a capsule 100 having a shell 110 is equipped with a strain gauge 120 mounted on the surface of the capsule shell 110, for example as illustrated in FIG. 1. Optionally, the strain gauge 120 senses stress and strains that the capsule experiences when passing through the gastrointestinal tract and which cause the capsule to move or prevent it from moving. In an exemplary embodiment of the disclosure, the strain gauge 120 is positioned internal to the capsule 100 on the inside wall of shell 110. Alternatively, the strain gauge 120 is positioned external to the capsule 100 on the outside wall of shell 110. In some embodiments of the disclosure, capsule 100 includes an image scanner 135 that uses radiation to scan and provide images of the position being scanned. Optionally, the image scanner 135 is selectively activated responsive to measurements taken by the strain gauge 120.

An exemplary strain gauge is manufactured by Vishay Precision Group, Inc from Raleigh, N.C. USA, for example the Micro Measurement SR-4 strain gauge. Alternatively, other compatible strain gauges may be used. Optionally, the strain gauge 120 is connected with appropriate electronics to control 130 that controls the functionality of capsule 100 by activating or deactivating the elements of capsule 100. In an exemplary embodiment of the disclosure, the appropriate electronics may include an amplifier, a Wein Bridge, an analog to digital converter and/or other electrical elements.

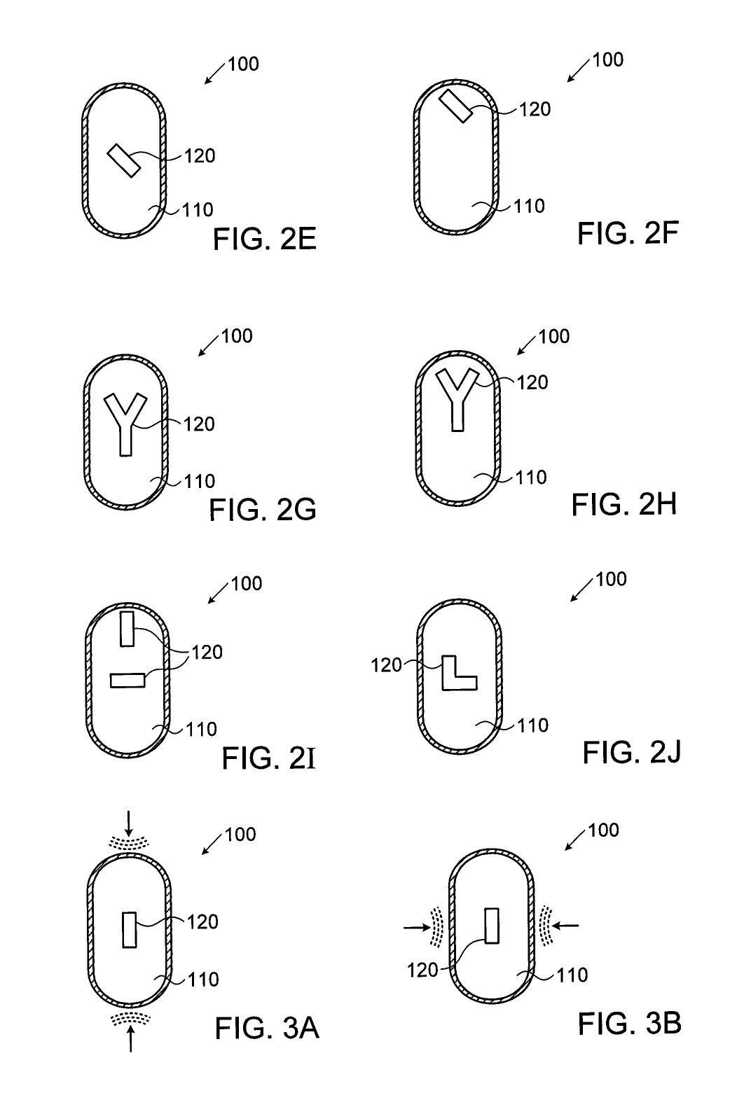

FIGS. 2A-2J show various possible positions and shapes for strain gauge 120. In an exemplary embodiment of the disclosure, capsule shell 110 is shaped as an elongated cylinder with domes on each end, and without having sharp edges. Alternatively, capsule shell 110 may be oval having an egg like shape or any other shape, for example shaped like a box. Optionally, capsule 100 may be implemented to include a combination of multiple strain gauges 120 in several different positions, orientations and/or shapes to aid in correctly detecting the source and nature of forces exerted on capsule 100 as it traverses through the gastrointestinal tract of a patient.

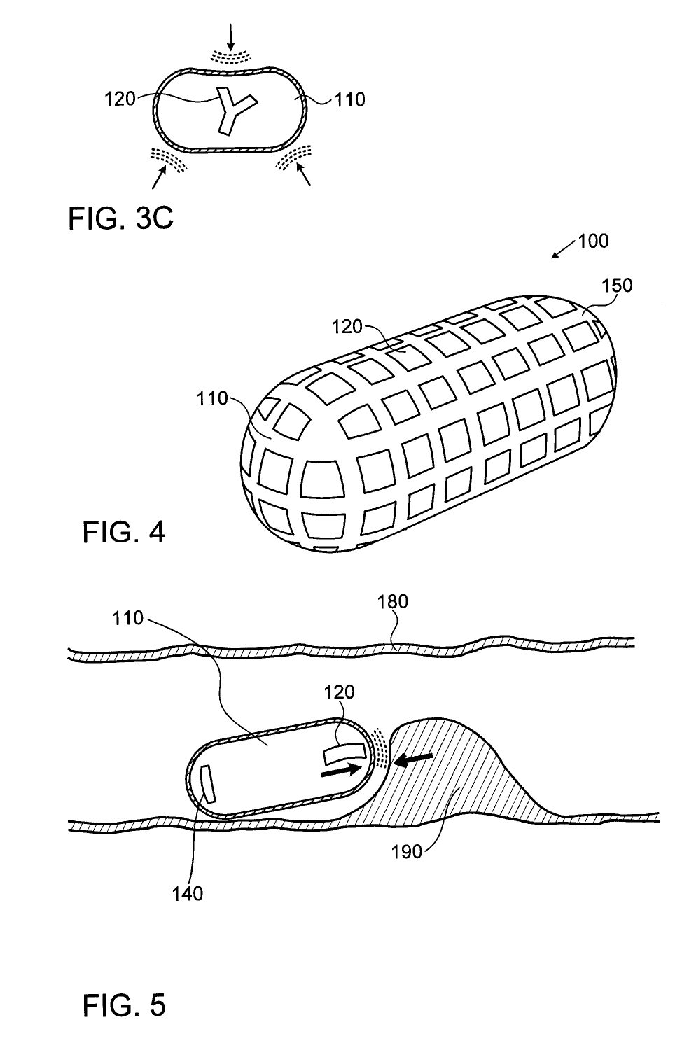

FIGS. 3A-3C are schematic illustrations of capsules with various strain forces applied to them, according to an exemplary embodiment of the disclosure. For example the forces may be applied to the ends of capsule 100, the sides of capsule 100 or various curve positions of shell 110. Optionally, the various strain forces depend on different physiological events that occur to capsule 100 on its route through the patient's body, for example passing through the stomach, the sphincters (e.g. esophageal sphincter, pyloric sphincter), the small intestine, the colon, the sigmoid and out through the rectum.

In an exemplary embodiment of the disclosure, capsule shell 110 is made of a semi-flexible material such as polycarbonate or similar plastic materials. Optionally, the width of the shell is less than 1 mm, for example between 0.2 mm to 0.7 mm. Optionally, the thickness of the shell allows it to slightly deform in response to external strain forces applying strain and/or stress on capsule shell 110. Optionally, capsule shell 110 essentially returns to its initial form when the strain forces are removed. In an exemplary embodiment of the disclosure, the strain forces are converted by strain gauge 120 to electrical signals that are transferred to control 130. Optionally, control 130 may transmit (170) the electrical signals to an external control 160 for analysis or provide a response internally responsive to the electrical signals, for example by initiating an image scan at the location in which the forces were applied or to record its relative or absolute location using a positioning system.

In an exemplary embodiment of the disclosure, analysis of the electronic signals can be used to determine a possible relevant physiological event causing the strain force, for example mass movement in the colon, passage through a sphincter and other events. In some embodiments of the disclosure, the determination based on strain gauge 120 may be combined with other measurements either taken inside the capsule or external to the patient's body to decide on taking actions with the capsule, for example using other sensors 144 that provide measurements such as a tilt sensor, a 3D accelerometer, or a 3D compass.

In some embodiments of the disclosure, capsule shell 110 is made of a soft material such as silicone or other similar material, so that small pressure on capsule 100 or movement of the capsule 100 can be sensed by strain gauge 120. Optionally, the shell may have a thickness of for example between 0.2 mm to 2 mm.

FIG. 4 is a schematic illustration of a capsule 100 with an array 150 of strain gauges 120 mounted on the internal or external surface of the capsule shell 110, according to an exemplary embodiment of the disclosure. Optionally, use of an array 150 of strain gauges 120 enables forming a pixilated sensing array of strain forces exerted on the outer surface of capsule 100. In an exemplary embodiment of the disclosure, an array arrangement can be used with a capsule shell from soft material or from semi-flexible material. Optionally, use of an array 150 of strain gauges can provide additional information related to the strain or stress exerted on the capsule, for example by following movement of the strain forces across the capsule shell 110. In an exemplary embodiment of the disclosure, use of an array of strain gauges 120 provides information that will be more accurate so that control 130 can be more accurate in starting and stopping image scanning by the capsule 100.

In an exemplary embodiment of the disclosure, the information provided by multiple strain gauges 120 from touching the colon walls, polyps or other deformations in the colon may be used to construct an image of the internals of the colon (e.g. polyps, deformations and other protrusions) with or without additionally using radiation to image the colon or by reducing the amount of scanning.

In some embodiments of the disclosure, the array 150 of strain gauges 120 is implemented by covering at least part of the exterior surface of the capsule shell 110 with an electronic tactile skin such as marketed by Sensor Products Inc. from Madison, N.J. U.S.A. Optionally, the tactile skin is connected to appropriate decoding electronics in control 130 to allow sensing touch information in the form of pressure measurements and pressure distribution on at least part of the capsule shell 110. Optionally control 130 or an external controller can use the touch information to determine whether capsule 100 is moving due to pressure of the colon walls on the exterior surface of the capsule shell 110.

In some embodiments of the disclosure, capsule 100 is designed to have a specific gravity (gram/cm3) higher than water (1 gram/cm3), for example 2 to 5 gram/cm3, so that the capsule 100 will sink in the colon content and specifically stay at the bottom of the cecum at the entry of the colon. When a mass movement occurs in the cecum, the capsule is practically squeezed by the colon muscles as they move the capsule from the cecum into the rest of the colon. Optionally, strain gauge 120 will sense this event due to the strain forces exerted on the capsule 100 and report it to control 130.

In some embodiments of the disclosure, a pressure sensor 140 is incorporated in the capsule 100, in addition to strain gauge 120. Optionally, the pressure sensor 140 measures hydrostatic pressure due to the content of colon 180. Optionally, control 130 receives measurements from strain gauge 120 and pressure sensor 140 to enhance the accuracy of the detection of polyps and other obstructions (e.g. a cancerous tissue mass). Control 130 may transmit (170) details of the position and the event to external control 160 to provide the information to a physician. Optionally, a physician can use the information regarding suspect positions for performing further examinations, such as a colonoscopy exam.

In an exemplary embodiment of the disclosure, the combination of at least one strain gauge 120 attached to the capsule shell 110 and the pressure sensor 140 helps to differentiate between pressures from the walls of the colon 180 and pressure from the content of the colon 180. The strain gauge 120 is used to sense strain on the capsule shell, for example strain that is applied by the colon walls along a cylindrical part of the capsule. Optionally, the pressure sensor 140 is located on an end of the capsule and is adapted to sense hydrostatic pressure of the colon content without influence of strains from the colon walls since the colon walls and the cylindrical shape of the capsule cause the capsule 100 to be aligned with its front and back ends in the direction of motion or opposite the direction of motion therefore the capsule end prevents strains from the colon walls to influence the pressure sensor.

Using two separate signals, one from the strain gauge 120 and one from the pressure sensor 140 enables control 130 or external control 160 to distinguish between strain events that occur due to the walls of the capsule pressing on the capsule, in which case only the strain gauge will sense these events, and events that are due to hydrostatic pressure of the content surrounding capsule 100 in which case, both the pressure sensor and the strain gauges may sense such pressures.

The ability of the control 130 to distinguish between these two types of events allows identifying when the walls of the colon are pressing the capsule and when hydrostatic pressure of the colon contents is causing the strain signals.

In an exemplary embodiment of the disclosure, combining the above information from the strain gauge 120 and pressure sensor 140 with position information from a position tracking system 142 (FIG. 1) allows off line analysis of the length and positions in the colon where the capsule was pressured by the colon wall, hence percent of colon coverage of where any obstructions are bulging from the walls of the colon 180.

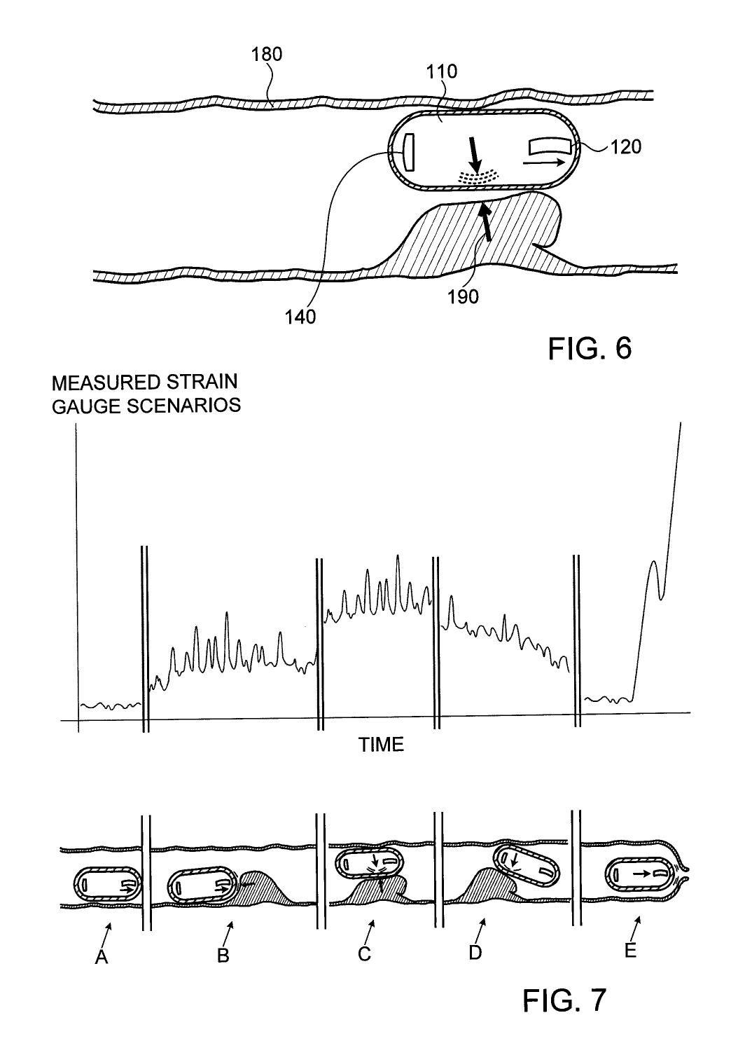

FIG. 6 is a schematic illustration of capsule 100 sliding over an obstruction 190, according to an exemplary embodiment of the disclosure. In an exemplary embodiment of the disclosure, capsule 100 may get caught in the lumen of the colon 180, for example between the walls of the colon 180 and various obstructions 190. Optionally, the exertion of pressure on the lumen walls cause baro-receptors in the colon to induce pressure waves through the colon at the obstruction location causing it to contract and expand to move the obstructing object forward (for example capsule 100). In an exemplary embodiment of the disclosure, strain gauge 120 and/or pressure sensor 140 will identify the contractions in different cases and allow control 130 to respond accordingly.

FIG. 7 is a schematic illustration of capsule 100 interacting with obstructions 190 in colon 180 versus the readings of strain gauge 120, according to an exemplary embodiment of the disclosure. Embodiment A illustrates capsule 100 in colon 180 without an obstruction. Embodiment B illustrates capsule 100 encountering obstruction 190. Embodiment C illustrates capsule 100 climbing over obstruction 190. Embodiment D illustrates capsule 100 descending from climbing over obstruction 190 to resume normal motion. Embodiment E illustrates capsule 100 at the end of the colon preparing to exit the colon.

In an exemplary embodiment of the disclosure, capsule 100 includes a position tracking system 142 (FIG. 1) such as an electromagnetic position tracking system so that the physician can be provided with positioning information that enables location of a suspect position for applying other tests.

In an exemplary embodiment of the disclosure, capsule shell 110 may be inflatable, for example like a balloon, so that it may be expanded in the colon to detect smaller obstructions or to stimulate the colon 180 to move the capsule 100. Optionally, capsule 100 can have an inflatable balloon or sack attached to it, so that they may be inflated while traversing through the colon to detect smaller obstructions and enhance stimulation of the colon 180 to react to the capsule.

In some embodiments of the disclosure, a hyper-osmolarity compound or other type of compound is used by a patient before swallowing capsule 100 to induce emptying or partial emptying of the colon. Optionally, as a result the diameter of the colon will be reduced to improve the probability of the capsule being obstructed by obstructions within the colon.

In some embodiments of the disclosure, the hyper-osmolarity compound may also serve as an X-Ray contrast agent, for example Telebrix Gastro that is marketed by Guerbet from Gorinchem, Nederlands. Optionally, the patient may intake the compound a few hours before swallowing the capsule 100 to enhance emptying the colon and to enhance the imaging ability of the scanner in capsule 100.

In some embodiments of the disclosure, obstructions 190 are detected solely based on the measurements of strain gauge 120. Alternatively a suspect location may be scanned with X-ray imaging to produce an image of the suspect location. Further alternatively or additionally, pressure sensor 140 may also be taken into consideration or other sensors 144 may be used, for example taking into account impedance changes by measuring the impedance in the vicinity of capsule 100 by placing electrodes on the exterior surface of capsule shell 110.

Additional measurements may include optical or near infra red changes close to the exterior surface of capsule shell 110, for example using optical or near infra red sensors. Optionally, capacitance changes near the exterior surface of capsule shell 110 may be detected using electrodes, since cancerous obstructions and normal tissue typically have different densities leading to differences in capacitance.

In an exemplary embodiment of the disclosure, a combination of multiple sensors is used to detect polyps or suspected lesions in colon 180, for example a combination of capsule tilt detected by the 3D accelerometer, change in the position of the capsule detected by the electromagnetic tracking system and the signals from at least one strain gauge 120 are combined to detect the possibility of encountering a polyp or a suspected lesion in the colon. During the capsule travel, the colon walls are typically close to the capsule pushing it along the colon, hence when a polyp or some other lesion is encountered, the capsule changes direction as it maneuvers around or above the polyp or lesion. A few millimeters later along the capsule tract the strain gauge 120 detects strong strain forces as the capsule 100 maneuvers around the polyp. Accordingly, based on the location in the colon 180, direction of travel and readings from the strain gauge 120 one can determine with a high likelihood regarding the presence of a polyp or some other lesion. This information is used by control 130 or external control 160 to notify a physician and/or activate the capsule scanning mechanism to record an image of the position.

In an exemplary embodiment of the disclosure, the signal provided by strain gauge 120 can be used to identify the exit of capsule 100 from the patients body, for example into a toilet. Optionally, a strong strain signal is recorded as a result of rectal pressure on capsule 100 as it exits. This signal is typically followed by a slow change in the signal of the strain gauge 120 as a result of a change in the surrounding temperature, for example changing from 36-37 C as in the body to a different temperature of the room (e.g. 20 C).

The sequence of signals may be used by control 130 or external control 160 to notify the patient that the capsule is out and also to initiate processes in the capsule such as sending the last data from the capsule to external control 160.

It should be appreciated that the above described methods and apparatus may be varied in many ways, including omitting or adding steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure.

It will be appreciated by persons skilled in the art that the present disclosure is not limited to what has been particularly shown and described hereinabove.

We claim:

1. A capsule for examining the gastrointestinal tract, comprising:
a capsule shell enclosing the capsule; wherein said capsule shell is designed to be swallowed by a user to traverse the user's gastrointestinal tract internally;
a strain gauge coupled to the capsule shell for measuring strain forces exerted on the capsule shell;
a control for receiving the measurements from the strain gauge and responding to the measurements.

2. A capsule according to claim 1, further comprising an image scanner that uses radiation to scan images of the surroundings of the capsule; wherein the image scanner is activated selectively by the control responsive to the measurements from the strain gauge.

3. A capsule according to claim 1, further comprising multiple strain gauges each monitoring a different area of the capsule shell.

4. A capsule according to claim 3, wherein said control follows movement of strain forces across multiple strain gauges.

5. A capsule according to claim 1, wherein said control identifies obstructions in the gastrointestinal tract responsive to the measurements from the strain gauge.

6. A capsule according to claim 1, wherein said control identifies obstructions in the gastrointestinal tract responsive to the measurements from the strain gauge and at least one other sensor.

7. A capsule according to claim 6, wherein the at least one other sensor is a pressure sensor that measures hydrostatic pressure due to the content surrounding the capsule.

8. A capsule according to claim 6, wherein the at least one other sensor is a position tracking system.

9. A capsule according to claim 6, wherein the at least one other sensor is an infra-red sensor.

10. A capsule according to claim 6, wherein the at least one other sensor is a 3D accelerometer.

11. A capsule according to claim 6, wherein the at least one other sensor is a 3D compass.

12. A capsule according to claim 1, wherein said capsule has a specific gravity greater than 1.

13. A capsule according to claim 1, wherein said strain gauge is coupled to the exterior of the shell capsule.

14. A capsule according to claim 1, wherein said strain gauge is coupled to the interior of the shell capsule.

15. A method of examining the gastrointestinal tract, comprising:
swallowing a capsule enclosed by a capsule shell;
measuring strain forces exerted on the capsule shell with a strain gauge coupled to the capsule shell;
transferring the measurements to a control; and
identifying obstructions from the measurements.

16. A method according to claim 15, further comprising:
selectively activating an image scanner that uses radiation to scan images of the surroundings of the capsule responsive to the measurements from the strain gauge.

17. A method according to claim 15, further comprising:
accepting measurements from at least one other sensor.

18. A method according to claim 17, wherein said other sensor is a pressure sensor that measures hydrostatic pressure due to the content surrounding the capsule.
